(19) United States
(12) Patent Application Publication
(10) Pub. No.: US 2018/0206475 A1
(43) Pub. Date: Jul. 26, 2018

(54) CHEMICAL APPLICATION DETECTION SYSTEM AND MOBILE VISUAL SENSING TECHNOLOGY

(71) Applicant: Mark A. Carter, Ankeny, IA (US)

(72) Inventor: Mark A. Carter, Ankeny, IA (US)

(21) Appl. No.: 15/891,844

(22) Filed: Feb. 8, 2018

Related U.S. Application Data
(63) Continuation-in-part of application No. 15/875,728, filed on Jan. 19, 2018.
(60) Provisional application No. 62/448,566, filed on Jan. 20, 2017.

(51) Int. Cl. A01M 7/00; B64D 1/18; G01N 33/00; B25J 11/00; B64C 39/02

(52) U.S. Cl. CPC A01M 7/0089; B64D 1/18; G01N 2021/6497; B25J 11/00; B64C 39/024; G01N 33/0098

(57) ABSTRACT

A chemical application detection system and mobile visual sensing technology utilizes drones, ground robotics, or fixed camera with a GPS system and a UV sensing camera. The detection device senses a color hue of one or more additives combined with a chemical treatment applied to a field above or below the canopy. The detected information is transmitted to a computer where the chemical treatment and location are displayed.

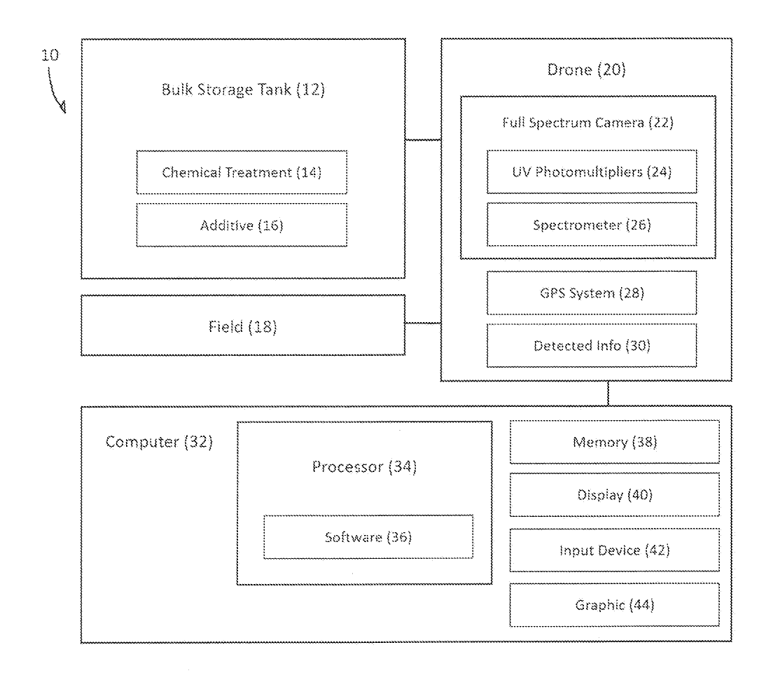

### CROSS REFERENCE TO RELATED APPLICATION

This application is a continuation-in-part of U.S. patent application Ser. No. 15/875,728 filed Jan. 19, 2018 which claims the benefit of U.S. Provisional Application No. 62/448,566 filed Jan. 20, 2017.

### BACKGROUND OF THE INVENTION

This chemical application detection system and mobile visual sensing technology invention is directed to a system for detecting the application of specific chemicals to a field, whether above or below the leaf canopy, and more particularly a system utilizing additives that emit ultraviolet (UV) spectrum light, to bulk chemicals applied to a field, that can be detected and identified by UV cameras mounted to various drones, ground robotics, or fixed cameras.

Presently, the application of chemicals to a field is an important process in enhancing crop growth, deterring the growth of weeds, and eliminate the presence of insects and other pests. There exist a number of ways to apply chemicals to a field, but there is no system for determining how well a field has been covered on top of the canopy, or how well the applied chemicals have penetrated through the canopy to the stalks and roots. As one example, agricultural drones are currently used to survey fields, buildings, and fence lines. Using a mounted camera and visible light, the agricultural drone is able to view crops and soil, view density and/or coverage (i.e. crop count, projected yields), and inspect areas for pest, flood, weed, or machine damage. Using infrared cameras, agricultural drones are able to detect a surface “reflectance” that absorbs and reflects ultraviolet (UV) solar rays not visible to the human eye. Plant leaves have a faint signature reflectance that is used to identify if a plant is healthy (i.e. absorbing or reflecting red light). As a result, current agricultural drone applications focus only on imaging visible or UV signatures of what is naturally occurring on crops and soil.

Needed is an agricultural application that is able to detect and differentiate various specific types of chemicals (herbicides, pesticides, etc.) present on a field from all levels of the plants from the top of the canopy, to the stalks and roots, as well as be able to display where and how much coverage of specific chemicals are present on a field. Also needed is a system that can detect chemical overspray beyond the perimeters of the field, and identify rogue plants needing to be removed. One solution is utilizing chemicals, vitamins, minerals, and fluorescents that naturally emit UV spectrum light as additives to agricultural chemicals, for the purpose of identifying, inspecting, and profiling what chemicals are actually on any given field, on top or below the leaf canopy, with drones, ground robotics, fixed-wing drones, or fixed cameras, equipped with a UV sensing camera. Also, a need exists as farms along rivers, creeks, or watershed areas being mandated to use UV additives so that farm chemicals from their fields can be monitored and identified. Requiring that UV color profile additives are mandatory in agricultural chemicals, governments, or large companies, could use drones or ground robotics to quickly inspect large areas of fields or farmlands to insure farm operators are using correct quantities of chemicals, not using harmful chemicals near watershed areas, or in areas with high concerns of overspray or run-off. Therefore, there exists a need in the art for a system that addresses these deficiencies.

An objective of the present invention is to provide a detection system that better monitors crops, soil, irrigation, and drainage to provide earlier detection than from the human eye.

Another objective of the present invention is to provide a detection system that assists in management of decisions in chemical application that also reduces chemical costs and better protect the environment.

These and other objectives will be apparent to those skilled in the art based upon the following written description, drawings, and claims.

### SUMMARY OF THE INVENTION

A chemical application detection system includes: 1) UV chemical detection additives. Utilizing chemicals, vitamins, minerals, and fluorescents that naturally emit UV spectrum light as additives to agricultural chemicals, for the purpose of identifying, inspecting, and profiling what chemicals are actually on any given field. This additive is mixed with the bulk chemicals. 2) A chemical mobile visual sensing technology includes various drones, ground robotics, or fixed cameras equipped with a GPS system and a UV sensing camera.

The mobile ultraviolet detection device senses different color hues from additives mixed with chemical treatments that have been applied to a field, how deeply the applied chemical penetrated the leaf canopy, and how long the applied chemical lasted on the plant.

The detected information is transmitted to a computer, downloaded, and stored. The computer processes the detected information and displays the dimensions of the field, the specific chemical treatments applied, and the location of the chemical treatments.

### BRIEF DESCRIPTION OF THE DRAWING

The FIGURE is a block schematic diagram of a chemical application detection system.

### DETAILED DESCRIPTION

Referring to the FIGURE, a chemical application detection system 10 includes a bulk storage tank 12 filled with a chemical treatment 14 such as a pesticide, a herbicide, a fertilizer, an algaecide, a fungicide, an insecticide, a sterilant or the like. Added to the chemical treatment 14 within the storage tank 12 is an additive 16 that naturally emits a UV spectrum light.

The additive 16 is of any type such as a benign or common chemical, mineral, fluorescent, or vitamin that emit different color hues in the UV spectrum that are undetectable to the human eye, but are detectible by various devices. Examples of some of the additives 16 are set forth in the table below.

| | | |
|---|---|---|
| Chemicals: | Quinine (blue) | Metaline |
| Minerals: | Europium (blue) | Calcite (red) |
| | Willemite (green) | Esperite (yellow) |
| | Clinohedrite (orange) | |
| Vitamins: | B2 (yellow) | Thiamine |
| | Niacin | Riboflavin |
| Fluorescent: | Calcein | |

A specific additive 16 or combination of additives 16 having a specific hue is assigned to a specific chemical treatment 14 and the mixture is packaged and sold for use. The specific additive 16 or combination creates a tag or unique UV colored signature that permits identification, inspection, and profiling.

The mixture is then applied to a field 18 in a conventional manner and the additive 16 naturally emits the specific hue associated with the chemical treatment 14 on a plant. Utilizing a detection device 20, such as a UAV drone, ground robotics, fixed-wing drone, fixed camera, or the like, an inspection of the field 18 is conducted. Preferably, the detection device is equipped with a UV camera 22 having ultraviolet photomultipliers 24 or a spectrometer 26 such as a hyper or multi spectrometer. Also, the detection device 20 is equipped with a GPS system 28.

In operation, the detection device 20, which is controlled manually or automatically, is directed to fly over the field 18, move under the canopy, or detect from a fixed position. As the detection device 20 inspects the field 18 the specific hue of the additive 16 is detected by the UV cameras. The detected information 30 includes the color of the hue and the location of where the hue is located. The detected information is then transmitted or downloaded to a computer 32 having a processer 34 that operates software 36, a memory 38, a display 40 and an input device 42. Using the transmitted/downloaded information 30, the software 36 of the computer produces a graphic 44 or digital display that is displayed and/or printed illustrating the dimension of the field 18 (or area) and the location of the various chemical treatments 14 preferably using a color that matches the hue of a specific additive 16.

Thus disclosed is a chemical application detection system and a mobile visual sensing equipment that can be used to determine if a field has been effectively covered by chemical treatments. The system is also used to inspect large areas to determine if farm operators are meeting mandatory chemical treatment requirements. For example, a determination could be made as to whether harmful chemicals are being used too near watersheds or in areas with concerns related to overspray and run-off.

Finally, the system is used to detect rogue plants. More specifically, instead of having individuals walk fields for hours, the system is adapted to focus on a single plant to detect, for example, an additive that adheres to a rogue plant and determine the rogue plant's location. Based on the location, the individual is directed to the location of the rogue plant for treatment or removal.

What is claimed is:

1. A chemical application detection system, comprising:
a mobile visual sensing technology adapted to detect information about one or more additives added to a chemical treatment and applied to a field.

2. The system of claim 1 wherein the mobile visual sensing technology device has UV sensing capability.

3. The system of claim 1 wherein the mobile visual sensing technology is equipped with a GPS system.

4. The system of claim 1 wherein the chemical treatment is selected from a group consisting of a pesticide, a herbicide, a fertilizer, an algaecide, a fungicide, an insecticide, and a sterilant.

5. The system of claim 1 wherein the one or more additives is selected from a group consisting of a chemical, a mineral, a vitamin, and a fluorescent.

6. The system of claim 1 wherein the one or more additives has a specific hue assigned to the chemical treatment.

7. The system of claim 1 wherein the detected information includes a color of a hue of the one or more additive and a location of where the hue is located.

8. The system of claim 1 wherein the detected information is transmitted to a computer where dimensions of a field and a location of one or more chemical treatments are displayed.

9. The system of claim 1 wherein the mobile visual sensing technology is ground robotics.

10. The system of claim 1 wherein the mobile visual sensing technology is adapted to detect information about a chemical treatment from under a leaf canopy.

11. The system of claim 1 wherein the mobile visual sensing technology is adapted to determine a location of a rogue plant.
